(19) United States
(12) Patent Application Publication
(10) Pub. No.: US 2021/0142643 A1
(43) Pub. Date: May 13, 2021

(54) PATIENT-FALL SCENARIO DETECTOR AND SYSTEMS AND METHODS FOR REMEDIATING FALL SCENARIOS

(71) Applicant: Smart Health Solutions LLC, Lakewood, NJ (US)

(72) Inventors: Eliezer Susna, Lakewood, NJ (US); Yaakov Weingarten, Pomona, NY (US)

(21) Appl. No.: 17/090,598

(22) Filed: Nov. 5, 2020

Related U.S. Application Data
(60) Provisional application No. 62/932,548, filed on Nov. 8, 2019.

(51) Int. Cl. G08B 21/04; A61B 5/11; A61B 5/00; A61B 5/01; G01J 5/10

(52) U.S. Cl. CPC G08B 21/043; G08B 21/0476; A61B 5/1117; A61B 5/1128; G01J 2005/0077; A61B 5/015; A61B 5/7264; G01J 5/10; A61B 5/746

(57) ABSTRACT

Methods for monitoring, and preferably mitigating, patient falls are provided. These methods may include generating a machine-learning library of possible fall scenarios. The methods may also include receiving a fall-alert condition from a sensor. The fall-alert condition may be based on a received fall scenario. The methods may also include generating a fall alert in response to receiving the fall-alert condition, logging the fall scenario and monitoring a response characteristic associated with the fall scenario and fall alert. Preferably the methods may include receiving fall scenario feedback from the monitoring and updating categorization of the logged fall scenario based on the fall-scenario feedback.

### CROSS-REFERENCE TO RELATED APPLICATION

This application is a non-provisional of U.S. Provisional Patent Application No. 62/932,548 filed on Nov. 8, 2019 and entitled “PATIENT-FALL SCENARIO DETECTOR AND SYSTEMS AND METHODS FOR REMEDIATING FALL SCENARIOS”.

### FIELD OF TECHNOLOGY

Aspects of the disclosure relate to detecting and preferably avoiding physically dangerous situations for nursing home patients and hospital patients.

### BACKGROUND OF THE DISCLOSURE

Many, if not the majority, of nursing home patients and hospital patients are physically challenged. One aspect of the physical challenges that face nursing home patients and hospital patients (collectively, “patient(s)”) is that the patients are in danger of falling. Furthermore, the danger of falling includes the danger of sustaining injury while falling.

Accordingly, it would be desirable to provide systems and methods that can detect a scenario in which a patient is in heightened danger of falling and/or sustaining an injury while falling. It would also be desirable to provide systems and methods for mitigating, where possible, the danger of falling and/or the possibility of sustaining an injury where falling.

It would be further desirable to provide systems and methods for detecting whether a person has fallen and/or is in need of immediate assistance.

### SUMMARY OF THE DISCLOSURE

A patient-fall scenario detector is provided. The detector may include a sensor having a thermal camera or other suitable device for monitoring a patient's position. An attachment mechanism may also be included for attaching the sensor to a pre-determined location. The detector may include a processor in electronic communication with the thermal camera. The detector may have access to a machine learning (“ML”) library. The ML library may be used to store a plurality of thermal-record-characterized fall scenarios. The processor may be configured to broadcast a fall-scenario alert signal when the thermal sensor detects a single patient position, or a series of patient positions, that is determined, by the processor electronic communication with the library, to correspond, at a level above a predetermined threshold correspondence level, to one of the stored plurality of thermal-record-characterized fall scenarios. The alert signal may comprise thermal camera and/or patient location information, and the processor may broadcast the alert signal to another pre-determined location, such as a caregiver's station. In some embodiments, the thermal sensor may be configured to continue to track, subsequent to detection of the single patient position or the series of patient positions, an outcome of the single patient position or the series of patient positions.

### BRIEF DESCRIPTION OF THE DRAWINGS

The objects and advantages of the disclosure will be apparent upon consideration of the following detailed description, taken in conjunction with the accompanying drawings, in which like reference characters refer to like parts throughout, and in which:

FIG. 1A shows a patient in a relatively safe, prone, position;

FIG. 1B shows the patient in a shifted position;

FIG. 1C shows the patient in a fall-critical position;

FIG. 1D shows the patient from a viewpoint associated with an over-the-bed sensor;

FIG. 2A shows a patient in a wheelchair;

FIG. 2B shows the patient shifting from a seated position to a possible fall position;

FIG. 2C shows the patient in a fall-danger position;

FIG. 3 shows an illustrative flow diagram of methods according to certain embodiments;

FIG. 4A shows a schematic representation of a patient in an 8×8 matrix of a thermal camera;

FIG. 4B shows a schematic representation of the patient in a 16×24 matrix of a thermal camera;

FIG. 4C shows a schematic representation of the patient in a 24×24 matrix of a thermal camera;

FIG. 4D shows a schematic representation of the patient in a 32×24 matrix of a thermal camera;

FIGS. 4E-4H show top-down schematic representations of the patient in the matrices shown in FIGS. 4A-4D;

FIGS. 4I-4L show matrices depicting visual information of a wheelchair-associated patient;

FIG. 5 shows exemplary algorithm steps that may be involved in systems and methods according to certain embodiments;

FIG. 6 shows exemplary circuitry for use in stepping down battery voltage in systems and methods according to certain embodiments;

FIG. 7 shows exemplary circuitry for use with an exemplary thermal camera in systems and methods according to certain embodiments;

FIG. 8A shows a schematic diagram of an exemplary WIFI chip (including processor) in systems and methods according to certain embodiments;

FIG. 8B shows exemplary circuitry for use in decoupling parts of the circuit from one another in systems and methods according to certain embodiments;

FIG. 8C shows exemplary circuitry for producing sound and/or light in accordance with the systems and methods described herein;

FIG. 9 shows exemplary parts and associated descriptions for use with certain embodiments of the systems and methods described herein;

FIG. 10 shows a top-down view of the sensor, showing component size schematics, according to exemplary embodiments;

FIG. 11 shows a bottom view of the sensor, showing component size schematics, according to certain embodiments;

FIG. 12 shows a first screen display of an exemplary mobile device according to certain embodiments;

FIG. 13 shows a second screen display of the exemplary mobile device according to certain embodiments;

FIG. 14 shows yet another screen display of the exemplary mobile device showing selectable vital statistics categories of a selected patient;

FIG. 15 shows an additional screen display of the exemplary mobile device showing a set of selectable vital statistics of a selected patient, the set different from that of the screen display shown in FIG. 14;

FIG. 16 shows a screen display of the readout associated with selected vital statistics data of a patient;

FIG. 17 shows an exemplary screen display of an 8×8 matrix (showing seven of the eight rows) for viewing by a member of the caregiving staff;

FIG. 18 shows an exemplary screen display depicting a patient outline and containing also an image of the displaying handheld mobile device;

FIG. 19 shows yet another exemplary screen display depicting patient data;

FIG. 20 shows an exemplary sensor with suitable electronic couplings used in systems and methods according to certain embodiments;

FIGS. 21-22 show examples of certain embodiments of a sensor according to certain embodiments;

FIGS. 23-24 show examples of certain embodiments of sensor positions and fields of view according to certain embodiments; and

FIGS. 25-26 show examples of certain embodiments of system and device structures according to certain embodiments.

### DETAILED DESCRIPTION OF THE DISCLOSURE

Aspects of the disclosure relate to a patient-fall scenario detector.

The system according to certain embodiments may include a sensor and an application (“app”). The system may be designed to obtain information about the patient, without use of bodily invasive equipment, nor with requiring the patient to wear any type of device on the patient's body.

The system may preferably be programmed to immediately alert and forward all pertinent medical information and important changes in the patient's condition to the medical and nursing staff, in an inexpensive and efficient manner. The system preferably operates absent any noticeable modifications in an existing building structure into which the system is installed.

The system preferably provides significant solutions to advance patient care provided by medical institutions, hospitals, nursing homes, etc. The system enables reaching and helping patients in a timely fashion. Specifically, the system enables providing proper immediate attention from medical, nursing and support staff in both emergency and non-emergency situations, saving lives and helping patients get the attention they need in a most timely fashion.

At the same time, the system preferably conserves resources of medical, nursing and other patient-care institutions because it may be configured to substantially continually service all the patients in the facility, thereby eliminating the need for extra staff to be used for routine room rounds.

Towards this goal, the system preferably includes a thermal heat sensor, according to certain embodiments, and a coordinating designated app.

In some embodiments, the system may include an active heat thermal sensor. The sensor preferably continuously screens and monitors the body movement. In certain embodiments, the sensor may also, in some embodiments, preferably screen and monitor patient's pulse, breathing and body movement. This information may be sent periodically or, in some embodiments, substantially constantly. For example, the information, such as body movement, breathing information and/or patient's pulse information, may be transmitted at a rate of 60 times per minute, to the system app.

Additionally, the system sensor preferably detects the patient's position in bed and identifies if a patient sits up and is about to fall—i.e., the system monitors for and detects potential possible fall scenarios.

In some embodiments, a connected mechanism on top of the sensor, or in some other suitable location, may preferably immediately shine a light on the patient and the patient's immediately surrounding area and sound an alarm in an effort to alert and/or arouse the patient and prevent the fall. This alarm, or a different alarm, also alerts staff in the area to come and help the patient. This fall prevention mechanism operates preferably independently of other systems and operates even in case of a power or internet outage.

Additionally, system wall and/or ceiling sensors can monitor, scan and/or detect any falling, for any reason, in any area, such as a room, hallway, shower or bathroom. Further, the system may be configured to detect a body in a fallen position without, necessarily, detecting the fall itself.

The system app may operate in tandem—i.e., in electronic communication—with the sensor. All information—e.g., body position and/or movement, pulse, breathing etc.—that is detected by the system sensor may be recorded, analyzed and saved in the app.

The app may be programmed and/or customized to each patient individually, and preferably automatically detects deviations from the patient's normal function and behavior. This information may be sent to the medical staff, regularly and/or on a need to know basis. In case of an extreme or unusual change, likely signifying a medical emergency, a high alert message may be released to the entire caregiving group on call. In addition, the app may regularly chart the patient according to a standard norm and/or to the patient's norm. It should be noted that, when the app has been programmed and/or customized to each patient individually, an initialization sequence may be implemented that pre-programs the app for the individual patient prior to actual deployment.

With each bed, room, hallway and/or bathroom preferably associated with its own individual sensor or sensors, the app may preferably parse the relevant information appropriately. It should be noted that typically any reference herein to a sensor should preferably refer to one or more sensors. More than one sensor may preferably provide additional information to the system because the additional sensors—to the extent that they are positioned at different locations vis-à-vis the patient—provide a greater depth to the retrieved information. Accordingly, multiple sensors may be used to triangulate the location of the patient using edge detection, bit-shrinking or other known techniques. All of these techniques will be more accurate and therefore more successful when leveraging multiple sensors instead of a single sensor. In certain embodiments, one or more of the foregoing sequences may be used to determine location of one or more body parts and the change of location of the body parts may be used to determine whether the patient has engaged in a fall-critical shift or is currently in a fall-critical position. The app may, in certain embodiments, notify the staff proximal to the area where the help is needed.

In some embodiments, different types of patient movement sensors may be combined to derive more complex and granular patient movement information. For example, patient movement sensors such as, for example, the patient movement sensors disclosed in detail herein, may be combined with information derived from one or more alternative types of patient movement sensors. Alternative types of patient movement sensors may include patient movement detection mats placed, for example, on a floor alongside the patient's bed. Thus, information derived from the sensors described herein in detail may be combined with information derived from patient movement detection mats or other suitable devices.

Besides receiving preferably all the information from the patients' sensors, other information regarding the patient's care—including patient's diet, patient's allergies, patient's medications and physician's notes—can be entered and recorded in the app.

Preferably all information regarding any registered patient can be retrieved both by the app when proximally located near the bed's sensor or by searching by patient's name in the general app. This can be useful in case of a patient who requires emergency medical attention.

Furthermore, the app may instruct the sensor to retrieve information in a variety of different vertically-arranged (or horizontally-arranged) levels. As such, the application may direct a ceiling mounted sensor to retrieve (or the app may directly retrieve from the sensor) information pertaining to the patient at a first level corresponding to the height of the bed. In addition, the application may also direct the ceiling mounted sensor to retrieve (or the app may directly retrieve from the sensor) information from the patient at a second level corresponding to the floor height. In this manner, the first set of derived information can correspond to a fall-danger detection scenario while the second set of derived information can correspond to a post-fall detection scenario whereby the patient is located on the floor in proximity to the bed. As such, a single sensor can be leveraged by an application to provide a variety of different areas of inspection.

It should be noted that the system may also be utilized to monitor such areas as hallways, bathrooms, lobbies, etc., as needed for the continued safety of the patient. One aspect of such monitoring may be that, because the transmitted signals may be of relatively low granularity vis-à-vis the patient image, the image produced may be of sufficiently low quality such that, while the data are completely adequate to monitor the patient's safety, the patient's privacy is protected and the patient's privacy concerns are respected.

In certain embodiments, the application should preferably be available to retrieve information regarding the medical history of the patient in response to a request from medical staff.

A patient-fall scenario detector is provided. The detector may include a thermal sensor for monitoring a patient's position. The detector may also include an attachment mechanism for attaching the sensor to a pre-determined location, such as, for example, a location on a wall or on a ceiling.

The detector may include a processor. The processor may be in electronic communication with the thermal sensor. The processor may also be in electronic communication with a machine learning (“ML”) library. The library may store a plurality of thermal-record-characterized fall scenarios.

The processor may be configured to broadcast a fall-scenario alert signal comprising thermal sensor and/or patient location information to a pre-determined location when the sensor (which may be a thermal sensor but is not required to be thermal; the sensor may be any relevant motion detector, such as radar) detects a patient position or a series of patient positions, that is determined, by the processor communication with the library, to correspond, at a level above a predetermined threshold correspondence level, to one of the stored plurality of thermal-record-characterized fall scenarios. It should be noted that the sensor may also be disposed on a wheelchair to monitor safety in situations where the patient is moved about using a wheelchair, yet has the capacity to lift himself or herself from the wheelchair and, thereby, may fall.

In addition, the thermal sensor may be configured to continue to track, subsequent to detection of the series of patient positions, an outcome of the series of patient positions.

The sensor preferably acts to continuously monitor pulse, breathing, heart rate, body temperature and room temperature. The sensor preferably adapts the measurements to the changing environment in which the occupant resides. For example, if temperature differences were created in the environment, or a fire broke out in the patient room, the sensor could detect it substantially immediately. Furthermore, if temperature differences were created because the occupant turned on an air conditioner, the sensor could detect this as well and would adjust its measurements accordingly.

In one embodiment, the sensor may be designed in the shape, and have the appearance and even the function, of a light fixture. Such light fixture can be mounted on the ceiling or on a wall or on any preferably pre-determined location. As such, the sensor, acting as a light fixture, may preferably be invisible to the user while at the same time providing the necessary information to the system—preferably with electronic communication between the sensor and processor. In some embodiments, the device may automatically turn on the light as well as the voice alert and/or other alarm alert systems when the patient's movements or other actions indicate a fall scenario. In certain embodiments, the alert system may also trigger an audible alert at the nurse's station or other relevant staff location. Alternatively, the alert system may trigger an alert at one or more mobile devices that are in the possession of staff.

In certain embodiments, the sensor may include a smart microphone/speaker. Such a device may preferably be open to programming by reprogramming the code of the device. For example, the care team can request the room temperature, heart rate, light on, light off and/or to retrieve a relevant medical file.

In some embodiments, the microphone can be selected to be highly sensitive. Such sensitivity may be leveraged to obtain a higher granularity of audio information from the patient. Such information may also contribute to more finely rendering the decision as to whether a fall scenario is in the process of occurring.

Certain embodiments may include a string of LED RGB (red/green/blue) bulbs as a part of the light fixture. Such bulbs may provide an additional indication of the patient's medical condition. For example, if the patient's heartbeat deteriorates, in addition to the audible alert, the lights on the sensor may appear as red lights.

When the sensor is on, and monitoring the patient's status, the bulbs may turn green. If there is no active WIFI connection and/or internet connection, the bulbs may turn blue. These are only examples of possible embodiments of use of the lights in electronic communication with the sensor.

With respect to turning off an alarming device, some embodiments of the sensor and/or the system including the sensor may include an infrared receiver for receiving a remote signal from an infrared broadcasting device. Such a signal may preferably be emitted by a handheld remote control device. Such a signal may preferably be operable to turn off the light and sounds associated with the alarming device. Such a signal may preferably also be operable to reset the device to a fully operational monitoring state.

In certain embodiments, the system may be configured to respond to detection of the fall scenario by further monitoring for a fall outcome associated with the series of patient positions.

In response to detecting the fall outcome associated with the series of patient positions, the thermal sensor may be further configured to monitor for a fall-with-injury outcome associated with the series of patient positions. The processor may be further configured to customize the thermal sensor to track the movements of a pre-determined patient.

In some embodiments, the detector may include a light. In some embodiments, the detector may be electronically coupled to a light. The light, when turned on, is configured to illuminate the area surrounding the patient. When the thermal sensor detects the series of patient positions that is determined to correspond, at a level above the predetermined threshold correspondence level, to one of the stored plurality of thermal-record-characterized fall scenarios, the detector turns on the light.

In some embodiments the system may include a second thermal sensor for monitoring a patient's position. Preferably, the first thermal sensor and the second thermal sensor are used in conjunction with one another to detect the series of patient positions that correspond to the one of the stored plurality of thermal-record-characterized fall scenarios.

Certain embodiments may include a method for monitoring, and preferably mitigating the occurrence of, patient falls. The method may include generating and/or storing a machine-learning library of possible fall scenarios.

The method may include receiving a fall-alert condition from one or more sensors. The receiving may include determining the fall-alert condition based on information derived from information received from the one or more sensors.

The fall-alert condition may be based on a received fall scenario. In response to the fall-alert condition the method may include generating a fall alert in response to receiving the fall-alert condition, logging the fall scenario, monitoring a response characteristic associated with the fall scenario and fall alert, receiving fall scenario feedback from the monitoring; and updating categorization of the logged fall scenario based on the fall scenario feedback.

In certain embodiments, methods may include assigning a fall-danger weight to each of the possible fall scenarios. In addition, the method may also include adjusting the alert, based on the fall-danger weight, of each of the possible fall scenarios. Alternatively, or in addition thereto, the methods may include adjusting the alert, based on the update of the categorization of the logged fall scenario, associated with the logged fall scenario.

The methods may also include weighting the fall alert vis-à-vis the logged fall scenario based, at least in part, on a value assigned for effectiveness of the response characteristic in remediating the logged fall scenario.

Apparatus and methods described herein are illustrative. Apparatus and methods in accordance with this disclosure will now be described in connection with the figures, which form a part hereof. The figures show illustrative features of apparatus and method steps in accordance with the principles of this disclosure. It is understood that other embodiments may be utilized, and that structural, functional, and procedural modifications may be made without departing from the scope and spirit of the present disclosure.

FIG. 1A shows a schematic diagram of a prone patient 102. The patient is lying in a bed 104. The bed includes partial rails 106 that are distributed at least a part of the way around the perimeter of the bed. In the exemplary bed 104 shown in the FIGURES, two rails are distributed alongside the head of the bed and two rails are distributed alongside the foot of the bed. Rails 106 preferably prevent the patient from falling out of bed 102.

Gap 108 is shown between two of rails 106. Gap 108 allows patient 102 to enter and exit bed 104. When a patient is physically challenged, gap 108 preferably enables caregivers to help patient 102 enter and exit bed 104.

However, gap 108 presents a danger to patient 102 when patient 102 is unattended. Because patient 102 can, without assistance, exit bed 104 through gap 108, this presents a danger of falling. Accordingly, it would be advantageous to patients and caregivers alike to provide a machine-learning (ML) based early-warning system that alerts medical, nursing and support staff when a patient has assumed a fall-critical position or has undergone a shift from one position to another, or has traversed a series of positions that indicates that the patient is in or is approaching a fall-critical scenario.

FIG. 1A shows one over-bed sensor 116 and an optional second sensor 118, according to certain embodiments of the disclosure. It should be noted that sensors 116 and 118 may include thermal imaging cameras as per the embodiments set forth herein. Sensors 116 and 118 may include any suitable device that may determine the position of the patient.

Sensor 116 preferably may monitor the patient from above the bed. It should be noted that sensor 116 may be the only sensor. Alternatively, sensor 118 may be the only sensor. In yet another alternative, both of sensors 116 and 118 may be used to determine the position of patient 102. Any suitable combination of one or more sensors may preferably be considered within the scope of the invention.

FIG. 1A shows patient 102 in a safe, prone position.

In FIG. 1B, patient 102 has shifted from position 110 to 112 and then shifted again to position 114. Position 114 may possibly be considered a fall scenario. As such, possible alarm 120 is shown as a possibility (in dashed lines). In such a circumstance, the determination as to whether an alarm condition exists depends on a review of an ML library. Such a review preferably determines whether, in past history, such a position or scenario has led to patient falls. If the position or scenario exceeds a preferably pre-determined threshold level of fall-danger, then the system may initiate an alarm, such as alarm 120, as well as any other mitigating actions.

FIG. 1C shows patient 102 in a fall-critical position 115. Associated with a patient position shown in FIG. 1C, an active alarm 122 is shown.

FIG. 1D shows patient 102 from a viewpoint associated with over-the-bed sensor 116 (not shown in FIG. 1D). FIG. 1D shows the patient shifting from position 110 to position 112 and then to position 114. Side of bed sensor 118 is shown. Possible alarm 120 is also shown (as in FIG. 1B, albeit from a different perspective).

Wheelchair bound patients also may shift from a sitting position to one or more positions that present a danger of falling. FIG. 2A shows a patient 202 in a wheelchair 204. A wheelchair-fixed sensor 206 is also shown.

FIG. 2B shows patient 202 shifting from a seated position 210 to a possible fall position 217. Possible alarm 220 is shown in FIG. 2B. It should be noted that, as in FIGS. 1B and 1D, the decision to alert the caregiving staff via alarm 220 preferably depends on a communication with an ML library to determine whether position 217 represents a historically dangerous position—either for the current patient or for a group of patients monitored currently or in the past.

FIG. 2C shows patient 202 in a fall-danger position 219. FIG. 2C also shows schematically alarm 222, which has been initiated in response to detection of fall-danger position 219.

FIG. 3 shows an illustrative flow diagram of methods according to certain embodiments. At step 301, the diagram shows using one or more detectors such as, for example, thermal camera(s) to monitor a patient's position. Step 303 shows detecting shifts of a patient's position to a fall-critical position—such a detection may be based at least in part on a the patient's history and/or a machine learning (ML) library of fall-critical and non-fall-critical positions.

Step 305 shows that the system may alert medical and/or support staff regarding shift of patient to a fall-critical position. Step 307 shows monitoring subsequent patient movement sequence (and/or response of staff) following fall-critical position.

Step 309 shows logging a record of staff response time. Logging may include logging staff response actions. Step 311 shows logging a fall/non-fall outcome as well as occurrence and/or severity of injury. Step 313 shows adding to ML library and logging veracity of detection—i.e., determining whether the fall-critical position was accurately assessed.

FIG. 4A shows a schematic representation of a patient in an 8×8 matrix of a thermal camera. In FIG. 4A, the patient is sitting up in bed in a questionably critical position. The 8×8 matrix shows the raw data that has been retrieved by the thermal camera. (It should be noted that patient depictions appearing in FIGS. 4A-4L are much sharper than would typically appear even in highly pixelated (e.g., 24×24) matrices. These figures have been shown herein in sharper relief than would be typically be viewed through the systems and methods of the invention, so as to illustrate the patient positions. FIG. 18 shows a more accurate depiction of how the patient would appear in a low-granularity display.)

FIG. 4B shows a schematic representation of a patient in a 16×24 matrix of a thermal camera. Because the representation in the 16×24 matrix is greater than the representation in the 8×8 matrix, the information contained therein is more granular, and more useful, in determining whether the patient is in a fall-critical position.

FIG. 4C shows a schematic representation of a patient in a 24×24 matrix of a thermal camera. FIG. 4D shows a schematic representation of a patient in a 32×24 matrix of a thermal camera.

FIGS. 4E-4H show top-down views of a patient in the positions shown in FIGS. 4A-4D. In addition, the matrices displayed in 4E-4H correspond, respectively, to the granularity of the matrices shown in FIGS. 4A-4D.

Furthermore, FIGS. 4I-4L shows matrices as depicting visual information derived from a wheelchair-based sensor. The matrices displayed in 4I-4L correspond, respectively, to the granularity of the matrices shown in FIGS. 4A-4D. (It should be noted that patient depictions appearing in FIGS. 4I-4L present the patient in the background behind a foreground wheelchair for purposes of clearly illustrating the patient positions.)

Each of FIGS. 4A-4L show matrices that are exemplary for use in systems and methods according to the embodiments. Preferably, these matrices may be displayed on one or more devices that are handheld by staff attending to the patient, in visible mounted displays, and/or in locations that are central to the location of the staff.

FIG. 5 shows exemplary algorithm steps that may be involved in systems and methods according to the embodiments. These steps may be implemented in the order that they are presented in FIG. 5 or in another suitable order. These steps include:

1. LOAD/GENERATE ML LIBRARY FOR POSSIBLE FALL SCENARIOS;

2. ASSIGNING A FALL DESIGNATOR VALUE TO EACH OF THE POSSIBLE FALL SCENARIOS;

3. GENERATE FALL SCENERIO/ALERT FROM THERMAL CAMERA SENSOR BASED ON FALL SCENARIO;

4. LOG FALL SCENARIO; FALL ALERT;

5. MONITOR ONE OR MORE RESPONSE CHARACTERISTIC(S);

6. MONITOR FALL SCENARIO/ALERT OUTCOME;

7. FEEDBACK FALL SCENARIO; RESPONSE CHARACTERISTICS; ALERT OUTCOME TO ML LIBRARY;

8. UPDATE CATEGORIZATION OF LOGGED FALL SCENARIO;

9. ADJUST FALL ALERT TO LOGGED FALL SCENARIO BASED ON UPDATE TO CATEGORIZATION OF LOGGED FALL SCENARIO; AND

10. WEIGHT ADJUSTMENT TO FALL ALERT VIS-A-VIS LOGGED FALL SCENARIO BASED, AT LEAST IN PART, ON EFFECTIVENESS OF RESPONSE CHARACTERISTIC(S) IN REMEDIATING LOGGED FALL SCENARIO CUSTOMIZATION STEP.

FIG. 6 shows an exemplary circuit 600 for use in stepping down the battery voltage—e.g., 3-5 volts—to a much lower voltage for use with the other circuits in the sensor. FIG. 6 also shows several LEDs for use as display LEDs in systems and methods according to the disclosure.

FIG. 7 shows circuitry for use with an exemplary thermal camera. In this particular embodiment, the thermal camera being used is a Panasonic AMG88 (8×8 matrix). It should be noted, however, that any suitable thermal camera, or other detector, may be used with systems and methods according to the disclosure.

FIG. 8A shows a schematic diagram of an exemplary WIFI chip (including processor) for use with various embodiments described in the disclosure. In FIG. 8A, the WIFI chip is an ESP32-WROOM-32 chip. However, it should be understood that the embodiments are not limited to this particular WIFI chip and that any other suitable WIFI chip may be used according to the embodiments set forth herein.

FIG. 8B shows circuitry for use in decoupling. Specifically, FIG. 8B shows decoupling capacitors. Decoupling capacitors are capacitors used to decouple one part of an electrical network (circuit) from another. Noise caused by other circuit elements is shunted through the capacitor, reducing the effect the noise has on the rest of the circuit. (It should be noted that DNP in the diagrams refers to “do not populate” and indicates that these parts are not needed to mount on the sensor, or, alternatively, the values represented therein are not necessarily specified for this application.)

FIG. 8C shows circuitry for producing sound and/or light in accordance with the systems and methods described herein.

FIG. 9 shows exemplary parts and associated descriptions for use with certain embodiments of the systems and methods described herein.

FIG. 10 shows a top-down view of a sensor, showing component size schematics, according to exemplary embodiments.

FIG. 11 shows a bottom view of the sensor, showing component size schematics, according to certain embodiments.

FIG. 12 shows a first screen display of a handheld mobile device to be used in certain embodiments.

FIG. 13 shows a second screen display of a handheld mobile device to be used in certain embodiments. Such a display typically lists various rooms where patients reside. The display of the rooms typically shows the name of the patient staying in the room, if the room is occupied.

FIG. 14 shows yet another screen, the screen displaying selectable vital statistics of a selected patient.

FIG. 15 shows an additional screen displaying a set of selectable vital statistics categories of a selected patient, the set different from that shown in FIG. 14.

FIG. 16 shows a screen displaying a readout associated with selected vital statistics data of a patient.

FIG. 17 shows an exemplary screen shot of an 8×8 matrix for viewing by a member of the staff (in this case, with seven rows visible on the screen).

FIG. 18 shows an exemplary screen displaying a patient outline and also its rough depiction on a handheld device.

FIG. 19 shows yet another exemplary depicting a rough patient outline on a display, below an 8×8 matrix of the patient's data.

FIG. 20 shows an exemplary sensor with suitable electronic couplings used in systems and methods according to the embodiments.

FIGS. 21-22 show views of examples of certain embodiments of a sensor according to the embodiments.

FIGS. 23-26 described below show examples of certain embodiments of a sensor according to the embodiments. These embodiments may preferably include a radar sensor and a thermal camera as sensors in the system. (FIG. 25 is not tied to any particular sensor structure or type.)

FIG. 23 shows a top-down room plan 2300. A device position 2302 is located, and indicated, in an exemplary position in the center of a rectangular-shaped room. Linear distances values given are in meters. The room includes exemplary dimensions of 7.0 meters by 2.5 meters.

Beds are located at positions 2304. A thermal camera field of view (FoV); which may be considered for the purposes of this application as a plane at an exemplary distance from the thermal camera of 2.5 meters, and substantially parallel to the ceiling/floor of the patient room) for viewing patient activity and/or patient positions is shown at 2306. A radar sensor FoV (which may be considered for the purposes of this application as a plane at an exemplary distance from the radar sensor of 2.5 meters, and substantially parallel to the ceiling/floor of the patient room) for deriving information from patient activity and/or patient positions is shown at 2308.

FIG. 24 shows various exemplary sensor FoVs for each of the sensors. Linear distances values given are in meters. Thermal camera FoV in X direction 2402 shows exemplary fields of view at distances of 2.00, 2.50 and 3.00 meters from the sensor. These distances correspond to horizontal distances of 5.67, 7.08 and 8.53 meters, respectively. Thermal camera FoV in Y direction 2404 shows exemplary fields of view at distances of 2.00, 2.50 and 3.00 meters from the sensor. These distances correspond to horizontal distances of 3.05, 3.81 and 4.60 meters, respectively.

Radar Sensor FoV in H-plane 2406 shows exemplary fields of view at distances of 2.50 and 3.00 meters from the sensor. These distances correspond to horizontal distances of 4.16 and 5.00 meters, respectively. Radar sensor FoV in E-plane 2408 shows exemplary fields of view at distances of 2.50 and 3.00 meters from the sensor. These distances correspond to horizontal distances of 2.59 and 3.10 meters, respectively.

FIG. 25 shows an exemplary system structure according to certain embodiments. A server that, among other tasks, stores data, is shown as 2502. Server 2502 may communicate with one or both of a web frontend 2504 (if one exists) and/or a mobile app. 2506 (which displays patient-states). This communication may be conducted using one or more of TCP/IP, Wifi, REST API and/or other protocols. In addition, server 2502 may communicate with a patient care device 2508 using one or more of TCP/IP, Wifi, REST API and/or other protocols. Patient care device 2508 may preferably be used, inter alia, to detect patient-state, provide light (such as with an LED lamp) and provide speaker control.

In certain embodiments communication may or may not be conducted using non-secure HTTPS.

FIG. 26 shows a schematic drawing of a sensor device structure 2600 according to certain embodiments. It should be noted that all the information contained in FIG. 26 is exemplary and, though it is directed to certain components, the components are exemplary and can be switched out for other suitable components.

A 12V/5A AC/DC converter 2602 is shown having a 12V connection with battery controller 2604. Battery controller 2604 is shown having a 12V connection with Lithium Ion reserve battery 2606. The device is powered normally with AC power supply, converting AC voltage to DC 12V. If AC/DC converter output voltage fall below 12V, reserve battery 2606 should connect automatically. As AC/DC converter output voltage recover to 12V, reserve battery 2606 should disconnect automatically and start charging.

A 5V/3A, 3.3V/2A DC/DC converter is shown as 2608. This is typically implemented to step down the battery voltage for use by the lower voltage components in the system. DC/DC converter 2608 is shown as having a 5V connection with computing unit 2610 which accepts a 5V/2A power source.

A 1.8<->3.3 logic level converter 2612 is shown for converting voltage to and from a level used for the heat camera 2614, which includes Shield 2616 and Array Breakout 2618, as well as radar sensor 2622. Radar sensor 2622 includes radar lens and holder 2620 which, in turn includes breakout board 2624 and sensor 2626. There is also shown an SPI coupling from radar sensor 2622 to converter 2612 and a USB connection to computing unit 2610.

Lighting and speaking module 2628 is also shown as electronically coupled to DC/DC converter 2608 as well as computing unit 2610. Lighting and speaking module 2628 may include all or some of speaker amplifier 2630, speaker 2632, logic level converter 2634, LED lamp RGB 2636, and/or LED lamp 2638.

The following are exemplary, and non-limiting, possible use cases, and algorithms related thereto, for possible implementations of sensors according to the disclosure.

Exemplary Use Cases:

Alarm Detecting:

When an issue is detected (patient shifts to or through fall-critical positions, patient falls on the floor, high patient temperature etc.), the device turns on the lights flashing, alarm sounds and alarm LEDs, and sends an Alarm State message to the Cloud Server.

Alarm State Turn Off:

When the device receives an “Alarm off” message by WiFi, it turns off the Alarm sound and LEDs. If no “Alarm off” message is received, but patient-state recovered to normal, in certain embodiments the device should turn the sound alarm off, and switch Alarm LEDs to “Warning state.”

Patient-State Measuring:

The device measures patient parameters periodically (e.g., once per second)—patient position, temperature, breathing rate and pulse rate. After measurement, the device sends the parameters to, in one exemplary embodiment, a Cloud Server. If one or more than one of the parameters is out of normal state template, in certain embodiments, the device generates the “Alarm detecting use case.”

Setting Up WiFi Connection:

If there is no Internet connection, device creates own WiFi Access Point and waiting for configuration. As Internet connection appears, device connects to the Cloud Server.

Patient Position Checking:

Device should check for next possible patient positions:
Patient lying on the bed (normal state, lights off)
Patient sitting on the bed (normal state, night light on)
Patient staying near the bed (normal state, night light on)
Patient lying on the floor (alarm state, lights on, alarm LEDs and sound)
Patient absent (normal state, lights off)

In the foregoing conditions, the patient position with alarm information should be sent to the Cloud Server.

Room Temperature Measuring:

The device should measure the room temperature and send it to the Cloud Server. Also, the device should use the device room temperature for tuning patient position detection algorithms. If the room temperature is out of pre-determined, normal limits, the device should send a warning message to the Cloud Server.

Patient Temperature Measuring:

The device should measure the patient temperature using the Thermal Camera and send it to the Cloud Server. If the patient temperature out of pre-determined, normal limits, the device should send a Warning Message to the Cloud Server and turn the Warning State on. If the patient temperature is out of critical limits, the device should send an Alarm Message to the Cloud Server and turn on the Alarm State.

Patient Breathing Rate Measuring:

The device should measure the patient breathing rate using the Thermal Camera and Radar Detector and send it to the Cloud Server. If the patient breathing rate is out of the normal limits, the device should send a Warning Message to the Cloud Server. If the patient breathing rate is at or beyond a pre-determined critical limit, the device should send an Alarm Message to the Cloud Server and turn on the Alarm State.

Patient Pulse Rate Measuring:

The device should measure the patient pulse rate using Radar Detector and send it to the Cloud Server. If the patient pulse rate is out of the normal limits, the device should send a Warning Message to the Cloud Server. If the patient pulse rate is at or beyond a pre-determined critical limit, the device should send an Alarm Message to the Cloud Server and turn on the Alarm State.

Alarm State On:

When the Alarm State turns on, the device sends an Alarm Message to the Cloud Server with information about alarm causes; and turns on the light, Alarm LEDs and Alarm Sound.

Alarm State Off:

When the Alarm State turns off by Mobile App, but the patient's state is still critical, the device turns off the light and Alarm Sound, but keeps Alarm LEDs on and sends a Confirmation Message to the Cloud Server.

When the Alarm State turns off by patient-state update, the device turns off the light, Alarm Sound and Alarm LEDs; and sends a new State Message to the Cloud Server.

Warning State On:

When a Warning State turns on, the device sends a Warning Message to the Cloud Server with information about warning causes; and, in certain embodiments, turns on the Warning LEDs.

Warning State Off:

When the Warning State turns off by Mobile App, but the patient's state is still critical, the device turns the Warning LEDs off and, in certain embodiments, sends a Confirmation Message to the Cloud Server.

When Alarm State turns off by patient-state update, the device turns the Warning LEDs off and sends a new State Message to the Cloud Server.

Light on:

When the Light turns on by Mobile App, the device turns on the Light and sends a Confirmation Message to the Cloud server.

Light Off:

When the Light turns off by Mobile App, the device turns off the Light and sends a Confirmation Message to the Cloud server.

Night Light on:

When the Night Light turns on by Mobile App, the device turns on the Night Light and sends a Confirmation Message to the Cloud server.

Night Light Off:

When the Night Light turns off by Mobile App, the device turns off the Night Light and sends a Confirmation Message to the Cloud server.

Alarm LEDs.

Alarm state

The Alarm LEDs Alarm State color is red.

Alarm LEDs. Warning state

The Alarm LEDs Warning State color is yellow.

Alarm LEDs. Normal state

The Alarm LEDs Normal State color is green.

Alarm Sound on:

The Alarm Sound can be a fixed pitch sound (C4-C4-C4) according ISO/IEC 60601-1-8. The Alarm Sound volume can be 50 dB.

Alarm Sound Off:

When the Alarm Sound turns off, the device stops sound generation and sends a Confirmation Message to the Cloud server.

Turning on Device:

When the device is switched on, it should check if it has a configuration for connecting to the internet via WiFi Access Point. If it does not, it should create its own WiFi Access Point to give a user a connection to it and to configure a WiFi connection.

After configuring device and connection to internet, the device should connect to the Cloud Server and send the current status to the server.

It should be noted that the foregoing shows and/or describes exemplary embodiments of systems and methods according to the embodiments.

The steps of methods may be performed in an order other than the order shown and/or described herein. Embodiments may omit steps shown and/or described in connection with illustrative methods. Embodiments may include steps that are neither shown nor described in connection with illustrative methods.

Illustrative method steps may be combined. For example, an illustrative method may include steps shown in connection with another illustrative method.

Apparatus may omit features shown and/or described in connection with illustrative apparatus. Embodiments may include features that are neither shown nor described in connection with the illustrative apparatus. Features of illustrative apparatus may be combined. For example, an illustrative embodiment may include features shown in connection with another illustrative embodiment.

The drawings show illustrative features of apparatus and methods in accordance with the principles of the invention. The features are illustrated in the context of selected embodiments. It will be understood that features shown in connection with one of the embodiments may be practiced in accordance with the principles of the invention along with features shown in connection with another of the embodiments.

One of ordinary skill in the art will appreciate that the steps shown and described herein may be performed in other than the recited order and that one or more steps illustrated may be optional. The methods of the above-referenced embodiments may involve the use of any suitable elements, steps, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

Thus, systems and methods for providing a patient-fall scenario detector and systems and methods for remediating fall scenarios are provided. Persons skilled in the art will appreciate that the present invention can be practiced by other than the described embodiments, which are presented for purposes of illustration rather than of limitation, and that the present invention is illustrated also by the following exemplary embodiments.

What is claimed is:

1. A patient-fall scenario detector, the detector comprising:
a thermal camera for monitoring a patient's position;
an attachment mechanism for attaching the thermal sensor to a first pre-determined location; and
a processor in electronic communication with:
the thermal sensor; and
a machine learning (“ML”) library, said library for storing a plurality of thermal-record-characterized fall scenarios;
wherein:
the processor is configured to broadcast a fall-scenario alert signal comprising thermal sensor and/or patient location information to a second pre-determined location when the thermal sensor detects a single patient position or a series of patient positions determined, by the processor electronic communication with the ML library, to correspond, at a level above a predetermined threshold correspondence level, to one of the stored plurality of thermal-record-characterized fall scenarios; and
the thermal sensor is configured to continue to track, subsequent to detection of the single patient position or the series of patient positions, an outcome of the single patient position or the series of patient positions.

2. The detector of claim 1, wherein the thermal sensor is further configured, in response to detecting the fall scenario associated with the single patient position or the series of patient positions, to monitor for a fall outcome associated with the single patient position or the series of patient positions.

3. The detector of claim 2, wherein, the thermal sensor is further configured, in response to detecting the fall outcome associated with the single patient position or the series of patient positions, to monitor for a fall-with-injury outcome associated with the single patient position or the series of patient positions.

4. The detector of claim 1, wherein the processor is further configured to customize the thermal sensor to track the movements of a pre-determined patient.

5. The detector of claim 1, further comprising a light, wherein:
the light is configured, when turned on, to illuminate the area surrounding the patient; and
when the thermal sensor detects the single patient position or the series of patient positions that is determined to correspond, at a level above the predetermined threshold correspondence level, to the one of the stored plurality of thermal-record-characterized fall scenarios, the detector turns on the light.

6. The detector of claim 1, wherein the thermal sensor is a first thermal sensor, and further comprising a second thermal sensor for monitoring a patient's position, wherein the first thermal sensor and the second thermal sensor are used together to detect the single patient position or the series of patient positions that correspond to the one of the stored plurality of thermal-record-characterized fall scenarios.

7. The detector of claim 1, further comprising a radar sensor, to be used in conjunction with the thermal sensor, for deriving information relating to the single patient position or the series of patient positions.

8. A method for monitoring, and mitigating, patient falls, said method comprising:
generating a machine-learning library of possible fall scenarios;
receiving a fall-alert condition from a sensor, said fall-alert condition based on a received fall scenario;
generating a fall alert in response to receiving the fall-alert condition;
logging the fall scenario;
monitoring a response characteristic associated with the fall scenario and fall alert;
receiving fall-scenario feedback from the monitoring; and
updating categorization of the logged fall scenario based on the fall-scenario feedback.

9. The method of claim 8 further comprising assigning a fall-danger weight to each of the possible fall scenarios.

10. The method of claim 9 further comprising adjusting the fall alert, based on the fall-danger weight, of each of the possible fall scenarios.

11. The method of claim 8 further comprising adjusting the fall alert, based on the update of the categorization of the logged fall scenario.

12. The method of claim 8 further comprising weighting the fall alert vis-à-vis the logged fall scenario based, at least in part, on a value assigned for effectiveness of the response characteristic in remediating the logged fall scenario.

13. The method of claim 8, wherein the receiving a fall-alert condition comprises receiving a fall-alert condition from a plurality of sensors.

14. The method of claim 8, wherein the receiving a fall-alert condition comprises receiving a fall-alert condition from information derived from a plurality of sensors.

15. The method of claim 8, wherein the receiving a fall-alert condition comprises receiving a fall-alert condition from a plurality of sensors, said plurality of sensors comprising a thermal sensor and a radar sensor.
